(19) United States
(12) Patent Application Publication
(10) Pub. No.: US 2007/0225320 A1
(43) Pub. Date: Sep. 27, 2007

(54) PROCESS FOR PREPARING CLOPIDOGREL

(76) Inventors: Eswaraiah Sajja, Hyderabad (IN); Raghupathi Reddy Anumula, Hyderabad (IN); Goverdhan Gilla, Hyderabad (IN); Lokeswara Rao Madivada, Krishna (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/691,295

(22) Filed: Mar. 26, 2007

Related U.S. Application Data
(60) Provisional application No. 60/807,783, filed on Jul. 19, 2006.

(30) Foreign Application Priority Data
Mar. 27, 2006 (IN) 545/CHE/2006

(51) Int. Cl. A61K 31/4743; C07D 498/02

(52) U.S. Cl. 514/301; 546/114

(57) ABSTRACT

A process for preparing clopidogrel or a salt thereof.

### INTRODUCTION TO THE INVENTION

The present invention relates to a process for the preparation of clopidogrel and intermediates thereof. In particular it relates a process for the preparation of methyl-α-amino-(2-chlorophenyl)acetate, an intermediate useful in the preparation of clopidogrel.

Clopidogrel bisulfate has a chemical name methyl (+)-(S)-α-(2-chlorophenyl)-6,7-dihydrothieno[3,2-c]pyridine-5(4H)-acetate sulfate, and is represented by structural Formula I.

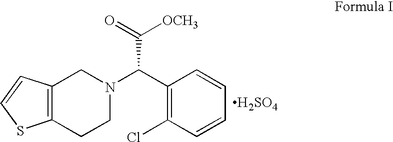

Clopidogrel is an inhibitor of ADP-induced platelet aggregation, acting by direct inhibition of the binding of adenosine diphosphate (ADP) binding to its receptor and of the subsequent ADP mediated activation of the glycoprotein GPIIb/IIIa complex.

The drug is currently being marketed in products sold as PLAVIX™ tablets containing about 98 mg clopidogrel bisulfate, which is the equivalent of 75 mg clopidogrel base. PLAVIX works by preventing platelets from sticking together to form clots that would restrict blood flow.

U.S. Pat. No. 4,847,265 discloses clopidogrel and its pharmaceutically acceptable salts.

U.S. Pat. No. 5,204,469 describes the preparation of a dextro-isomer of methyl α-(4,5,6,7-tetrahydro-5-thieno[3,2-c]pyridyl)(2-chlorophenyl)acetate.

International Application Publication No. WO 2006/003671 A1 describes a process for resolution of α-amino-(2-chlorophenyl)-acetic acid methyl ester.

U.S. Pat. No. 6,812,363 B2 discloses a process for the racemisation of (−)-α-amino-(2-chlorophenyl)-acetic acid methyl ester.

U.S. Pat. No. 6,429,210 discloses crystalline Form I and Form II of clopidogrel hydrogen sulfate.

The aforementioned processes use either mixtures of solvents or involve a large number of steps, thus leading to increases in the processing time cycle, and rendering the process expensive and unsuitable for use on an industrial scale.

Hence, there is a need to provide a simple and improved process for the preparation of α-amino-(2-chlorophenyl)-acetic acid methyl ester, which is economical, ecofriendly and reproducible with high yield and purity.

### SUMMARY OF THE INVENTION

The present invention relates to a process for the preparation of clopidogrel and intermediates thereof.

In an aspect, the present invention relates to a process for the preparation of methyl-α-amino-(2-chlorophenyl)-acetate of Formula V comprising:

a) reacting methyl-α-amino-(2-chlorophenyl)-acetate of Formula V with L-(+)-tartaric acid;

b) separating the formed tartaric acid salt of S-(+)-methyl-α-amino-(2-chlorophenyl)-acetate of Formula III from a reaction mixture; and

c) heating the reaction mixture to form methyl-α-amino-(2-chlorophenyl)-acetate of Formula V.

In another aspect, the present invention relates to a process for the preparation of clopidogrel or a salt thereof comprising:

a) reacting a tartaric acid salt of S-(+)-methyl-α-amino-(2-chlorophenyl)-acetate of Formula III with a suitable base to form S-(+)-methyl-α-amino-(2-chlorophenyl)-acetate of Formula II;

b) reacting S-(+)-methyl-α-amino-(2-chlorophenyl)-acetate of Formula II with 2-thienyl-p-toluenesulfonate to form S-(+)-methyl-α-(2-thienylethylamino)-(2-chloro phenyl)acetate hydrochloride of Formula VIII; and

c) reacting S-(+)-methyl-α-(2-thienyl ethyl amino)-(2-chloro phenyl)acetate hydrochloride of Formula VIII with formaldehyde to form clopidogrel.

An embodiment of the invention provides a process for preparing clopidogrel or a salt thereof, comprising reacting racemic methyl alpha-amino-(2-chlorophenyl)acetate with L-(+)-tartaric acid, separating a formed tartaric acid salt of S-(+)-methyl alpha-amino-(2-chlorophenyl)acetate from a reaction mixture, and heating a reaction mixture to form racemic methyl alpha-amino-(2-chlorophenyl)acetate.

Another embodiment of the invention provides a continuous process for preparing clopidogrel or a salt thereof, comprising reacting racemic methyl alpha-amino-(2-chlorophenyl)acetate with L-(+)-tartaric acid, separating a formed tartaric acid salt of S-(+)-methyl alpha-amino-(2-chlorophenyl)acetate from a reaction mixture, heating a reaction mixture to form racemic methyl alpha-amino-(2-chlorophenyl)acetate, and reacting formed racemic methyl alpha-amino-(2-chlorophenyl)acetate with additional L-(+)-tartaric acid to form a tartaric acid salt of S-(+)-methyl alpha-amino-(2-chlorophenyl)acetate.

In a further embodiment, the invention provides a process for the preparation of 2-thienyl-p-toluenesulfonate by reacting thiophene-2-ethanol with a p-toluene sulfonyl halide in a solvent comprising toluene or dichloromethane.

The processes of present invention are simple, cost effective, ecofriendly, robust, reproducible and well suited to be used on an industrial scale.

### BRIEF DESCRIPTION OF THE DRAWING

FIG. 1 is a schematic representation of processes of the present invention.

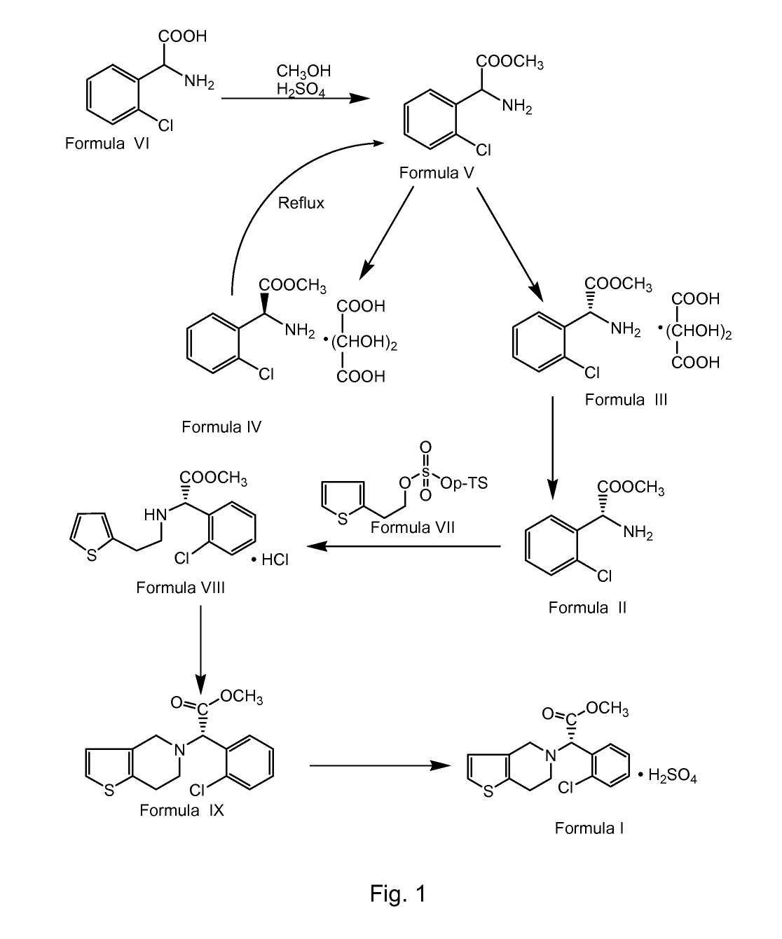

### DETAILED DESCRIPTION OF THE INVENTION

The present invention relates to a process for the preparation of clopidogrel and intermediates thereof

In an aspect, the present invention relates to a process for the preparation of methyl-α-amino-(2-chlorophenyl)-acetate of Formula V, comprising:

a) reacting methyl-α-amino-(2-chlorophenyl)-acetate of Formula V with L-(+)-tartaric acid;

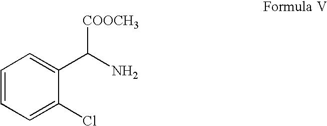

b) separating a formed tartaric acid salt of S-(+)-methyl-α-amino-(2-chlorophenyl)-acetate of Formula III from a reaction mixture; and

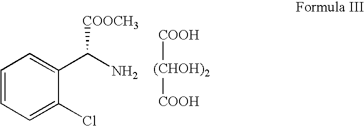

c) heating the reaction mixture to form methyl-α-amino-(2-chlorophenyl)-acetate of Formula V.

Step a) involves reaction of methyl-α-amino-(2-chlorophenyl)-acetate of Formula V with L-(+)-tartaric acid in a suitable solvent.

Suitable solvents include but are not limited to: alcohols such as methanol, ethanol, propanol, butanol and the like; ketonic solvents such as acetone, ethyl methyl ketone, isobutyl ketone and the like; nitrile solvents such as acetonitrile and the like; and mixtures thereof or their combinations with water in various proportions without limitation.

Suitable alternative chiral acids that can be used include but are not limited to L-(+)-mandelic acid, S-(+)-camphor sulfonic acid and the like.

The reaction can be carried out at any temperature ranging from about −10° C. to about 50° C.

Optionally a small quantity of S-(+)-methyl-α-amino-(2-chlorophenyl)-acetate of Formula III can be added as seed crystals.

Step b) involves separating the tartaric acid salt of S-(+)-methyl-α-amino-(2-chlorophenyl)acetate of Formula III from the reaction mixture.

The methods by which the solid material is recovered from the reaction mixture, with or without cooling below the operating temperature, can be any of techniques such as filtration by gravity, or by suction, centrifugation, decantation and the like. If desired, the crystals can be washed with a solvent to wash out the mother liquor.

The wet cake obtained above optionally may be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods such as from about 1 to 20 hours, or longer, to achieve a desired purity.

The mother liquor containing a reaction mixture, obtained from step b), is typically used in the next step directly or it can be concentrated to a suitable volume to get optimum yield and purity.

Step c) involves heating the reaction mixture to form methyl-α-amino-(2-chlorophenyl)-acetate of Formula V.

Heating can be carried out at temperatures of about 30° C. to about 100° C. or about 60° C. to about 80° C., or at the reflux temperature of the solvent used.

After heating the reaction mixture for desired time periods, such as from about 1 to 36 hours, or about 1 to 15 hours, the solvent is removed from the reaction mixture by a suitable technique to obtain a residue.

Removal of the solvent may be carried out suitably using evaporation, atmospheric distillation, or distillation under vacuum.

Distillation of the solvent may be conducted under a vacuum, such as below about 100 mm Hg to below about 600 mm Hg, at elevated temperatures such as about 20° C. to about 70° C. Any temperature and vacuum conditions can be used as long as there is no increase in the impurity levels of the product.

Suitable techniques which can be used for the solvent removal include distillation using a rotational evaporator device such as a Buchi Rotavapor, spray drying, agitated thin film drying (“ATFD”), and the like.

The residue is suspended in a mixture of water and an organic solvent and pH is adjusted to about 5 to about 8, using a suitable base.

Suitable bases that can be used include but are not limited to: inorganic bases such as sodium hydroxide, potassium hydroxide, sodium carbonate, potassium carbonate, sodium bicarbonate, potassium bicarbonate, sodium methoxide, potassium methoxide and the like; and organic bases such as methyl amine, ethyl amine, triethylamine, pyridine and the like.

After completion of pH adjustment, the product is extracted into the organic solvent and the solvent is removed to obtain the product.

Suitable organic solvents include but are not limited to: hydrocarbons such as n-hexane, n-heptane, cyclohexane, toluene, xylene and the like; halogenated solvents such as dichloromethane, ethylene dichloride and the like; and mixtures thereof.

Removal of the solvent may be carried out suitably using evaporation, atmospheric distillation, or distillation under vacuum.

Distillation of the solvent may be conducted under a vacuum, such as below about 100 mm Hg to below about 600 mm Hg, at elevated temperatures such as about 20° C. to about 70° C. Any temperature and vacuum conditions can be used as long as there is no increase in the impurity levels of the product.

Suitable techniques which can be used for the distillation include, distillation using a rotational evaporator device such as a Buchi Rotavapor, spray drying, ATFD and the like.

In another aspect, the present invention relates to a process for the preparation of clopidogrel or a salt thereof, comprising:

a) reacting the tartaric acid salt of S-(+)-methyl-α-amino-(2-chlorophenyl)acetate of Formula III with a suitable base to form S-(+)-methyl-α-amino-(2-chlorophenyl)acetate of Formula II;

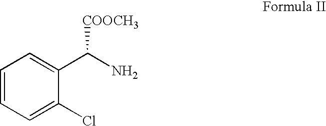

b) reacting S-(+)-methyl-α-amino-(2-chlorophenyl)acetate of Formula II with 2-thienyl-p-toluenesulfonate of Formula VII to form S-(+)-methyl-α-(2-thienylethylamino)-(2-chlorophenyl)acetate hydrochloride of Formula VIII; and

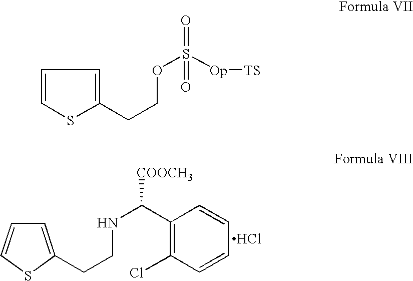

c) reacting S-(+)-methyl-α-(2-thienylethylamino)-(2-chlorophenyl)acetate hydrochloride of Formula VIII with formaldehyde to form clopidogrel of Formula IX.

Step a) involves reacting the tartaric acid salt of S-(+)-methyl-α-amino-(2-chlorophenyl)acetate of Formula III with a suitable base to form S-(+)-methyl-α-amino-(2-chlorophenyl)acetate of Formula II;

The tartaric acid salt of S-(+)-methyl-α-amino-(2-chlorophenyl)acetate of Formula III can be obtained by the process described above or from any other suitable processes.

Suitable bases that can be used include but are not limited to: inorganic bases such as sodium hydroxide, potassium hydroxide, sodium carbonate, potassium carbonate, sodium bicarbonate, potassium bicarbonate, sodium methoxide, potassium methoxide and the like; organic bases such as methylamine, dimethylamine, triethylamine, di-isopropylamine N,N-di-isopropylethylamine, butyl amine and the like; and mixtures thereof.

Step b) involves reacting S-(+)-methyl-α-amino-(2-chlorophenyl)acetate of Formula II with 2-thienyl-p-toluenesulfonate to form S-(+)-methyl-α-(2-thienylethylamino)-(2-chlorophenyl)acetate hydrochloride of Formula VIII.

Suitable solvents that can be used in step b) include but are not limited to water, tertiary-butyl acetate, toluene and the like.

Reaction is carried out at temperatures of about 30° C. to about 120° C., or about 80° C. to about 110° C., or at the reflux temperature of the solvent used.

After heating the reaction mixture for desired time periods from about 1 to 40 hours, or about 25 to 35 hours, the solvent is removed from the reaction mixture by a suitable technique to obtain a residue.

Removal of the solvent may be carried out suitably using evaporation, atmospheric distillation, or distillation under vacuum.

Distillation of the solvent may be conducted under a vacuum, such as below about 100 mm Hg to below about 600 mm Hg, at elevated temperatures such as about 20° C. to about 70° C. Any temperature and vacuum conditions can be used as long as there is no increase in the impurity levels of the product.

Suitable techniques which can be used for the distillation include distillation using a rotational evaporator device such as a Buchi Rotavapor, spray drying, ATFD and the like.

The residue is suspended in a mixture of water and an organic solvent and the product is extracted into the organic solvent.

The organic layer containing the product can be treated with a suitable acid such as hydrochloric acid, hydrobromic acid, acetic acid and the like to convert the product into a corresponding acid addition salt. The hydrochloride salt is shown as Formula VIII.

Step c) involves reacting the S-(+)-methyl-α-(2-thienylethylamino)-(2-chlorophenyl)acetate acid addition salt, such as the hydrochloride of Formula VIII, with formaldehyde to form clopidogrel.

Formaldehyde that is used in step c) can be in the form of aqueous or organic solutions. In one embodiment aqueous formaldehyde solutions having concentrations of about 40% w/v is used.

A suitable volume of formaldehyde ranges from about 3 L, to about 15 L, or about 5 L, per kg of the compound of Formula VIII.

The reaction is carried out at temperatures about 10° C. to about 50° C., or about 20° C. to about 30° C., for desired time periods from about 1 to 30 hours, or about 20 to 25 hours, to achieve reaction completion. The time required will depend on the chosen conditions.

After completion of the reaction the unwanted solid materials are removed form the reaction mixture, such as by filtration.

A suitable organic solvent is added to the clarified solution and pH is adjusted to about 5 to about 8 with a suitable base.

Suitable bases include but are not limited to: organic bases such as methylamine, dimethylamine, triethylamine, di-isopropylamine N,N-di-isopropylethylamine, butylamine and the like; inorganic bases including alkali metal compounds such as sodium hydride and potassium hydride, sodium hydroxide, potassium hydroxide, sodium methoxide, potassium methoxide, sodium carbonate, sodium bicarbonate, potassium carbonate, potassium bicarbonate and the like; and mixtures thereof.

After completion of pH adjustment, the product is extracted into the organic solvent and the solvent is removed to obtain the product.

Suitable organic solvents include but are not limited to: hydrocarbons such as n-hexane, n-heptane, cyclohexane, toluene, xylene and the like; halogenated solvents such as dichloromethane, ethylene dichloride and the like; and mixtures thereof.

Removal of the solvent may be carried out suitably using evaporation, atmospheric distillation, or distillation under vacuum.

The obtained clopidogrel can be converted into its bisulfate salt of Formula I by reacting with sulfuric acid in acetone or isobutyl alcohol, or aqueous mixtures thereof.

In yet another aspect the present invention relates to a process for the preparation of 2-thienyl-p-toluenesulfonate by reacting thiophene-2-ethanol with a p-toluene sulfonyl halide in a solvent comprising toluene or dichloromethane.

Suitably the reaction is conducted in the presence of a base such as: inorganic bases including sodium hydroxide, potassium hydroxide, sodium carbonate, potassium carbonate, sodium bicarbonate, potassium bicarbonate, sodium methoxide, potassium methoxide and the like; and organic bases such as methylamine, ethylamine, triethylamine, pyridine and the like.

Clopidogrel or its pharmaceutically acceptable salts prepared in accordance with the present invention contain less than about 0.5%, or less than about 0.1%, of any one or more of the corresponding impurities like clopidogrel bisulfate impurity A, impurity B, and impurity C, as characterized by a high performance liquid chromatography (“HPLC”) chromatogram obtained from a mixture comprising the desired compound and one or more of the said impurities. The percentage here refers to weight percent obtained from the area-% of the peaks representing the impurities. Clopidogrel and salts thereof also are substantially free of other process-related impurities

As used herein, “clopidogrel bisulfate impurity A” refers to (+)-(S)-(o-chlorophenyl)-6,7-dihydrothieno[3,2-c]pyridine-5-(4H)acetic acid represented by Formula Ia;

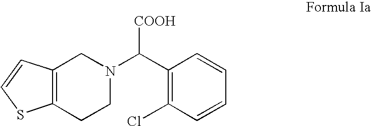

“clopidogrel bisulfate impurity B” refers to Methyl (±)-(o-chlorophenyl)-4,5-dihydrothieno[2,3-c]pyridine-6-(7H)-acetate, hydrogen sulfate represented by Formula Ib; and

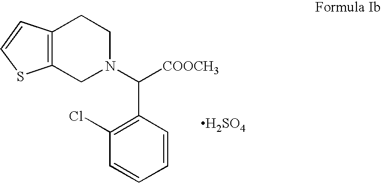

“clopidogrel bisulfate impurity C” refers to Methyl (−)-(R)-(o-chlorophenyl)-6,7-dihydrothieno[3,2-c]pyridine-5-(4H)acetate, hydrogen sulfate represented by Formula Ic.

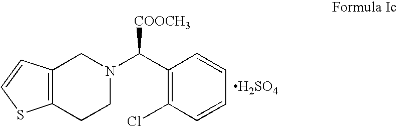

Clopidogrel or its salts obtained in the present invention contain less than about 5000 ppm, or less than about 3000 ppm, or less than about 1000 ppm, or less than about 200 ppm, or less than about 100 ppm, of individual residual organic solvents or organic volatile impurities.

Clopidogrel or its salts obtained in the present invention contain less than about 4000 ppm of acetone, or less than about 2000 ppm, or less than about 200 ppm of isopropanol, less than about 200 ppm, or less than about 100 ppm of methanol, and less than about 25 ppm of mesityloxide and less than about 15 ppm of dichloromethane, or less than about 5 ppm of acetone, ethyl acetate, and tertiary-butyl acetate.

Pharmaceutically acceptable acid addition salts of clopidogrel obtained according to the process described in this invention have a bulk density of less than about 0.75 g/ml or less than about 0.65 g/ml before tapping, and bulk density of less than about 2 g/ml, or less than about 1 g/ml after tapping. The bulk densities are determined using Test 616 “Bulk Density and Tapped Density,” United States Pharmacopeia 24, United States Pharmacopeial Convention, Inc., Rockville, Md., 1999, pages 1913-4).

The processes of present invention are simple, cost effective, easily reproducible, and eco-friendly, producing the desired compounds with high yield and purity.

Certain specific aspects and embodiments of the present invention will be explained in more detail with reference to the following examples, which are provided by way of illustration only and should not be construed as limiting the scope of the invention in any manner.

### EXAMPLE 1

### Preparation of α-Amino-(2-Chlorophenyl)-Acetic Acid Methyl Ester (Formula V)

900 liters of methanol and 180 kg of DL-2-chlorophenylglycine of Formula VI were charged into a clean and dry reactor. 145.3 kg of concentrated sulphuric acid was added slowly to the above reaction mixture below 40° C. over 3.5 hours, followed by heating to about 67.5° C. The resultant reaction mixture was stirred at about 67.5° C. for about 15 hours. After completion of the reaction, the solvent was distilled completely at about below 70° C. under vacuum. 540 liters of water was charged to the residue followed by stirring for about 30 minutes. The mixture was cooled to about 25° C. followed by charging of 360 liters of methylene chloride. The resultant reaction mass was stirred at about 25° C. for about 10 minutes followed by cooling to a temperature of about 10° C. Reaction mass pH was adjusted to about 7.62 by the addition of 360 liters of 30% aqueous sodium carbonate at below 20° C. over about 3.5 hours. The resultant reaction suspension was stirred below 20° C. for about 30 minutes. The layers were allowed to settle for about 45 minutes and the bottom organic layer was separated. The aqueous layer was extracted with 2×90 liters of methylene chloride followed by separation of organic and aqueous layers. All the organic layers were combined and washed with 180 liters of water. Organic and aqueous layers were separated and the organic layer was distilled completely below 70° C. under vacuum to afford 140 kg (yield: 72.35%) of the title compound.

### EXAMPLE 2

### Preparation of L-(+)-Tartaric Acid Salt of α-Amino-(2-Chlorophenyl)Acetic Acid Methyl Ester (Formula III)

1485 liters of methanol and 140 kg of α-amino-(2-chlorophenyl)acetic acid methyl ester of Formula V were charged into a clean and dry reactor followed by stirring for about 10 minutes. 105 kg of L-(+)-tartaric acid was charged to the above reaction solution at about 25° C. followed by stirring for about 10 minutes. 1.4 kg of S-(+)-isomer of amino-(2-chloro-phenyl)acetic acid methyl ester of Formula III was charged as seeding crystals to the reaction mass followed by stirring for about 10 minutes. The obtained reaction mass was cooled to about 3° C. followed by stirring for about 2.5 hours. The solid that separated was centrifuged followed by subjecting to spin drying for about 30 minutes to afford 117 kg of wet solid title compound. 280 liters of methanol was charged into a clean and dry reactor followed by the charging of the above obtained wet solid. The suspension was stirred at about 30° C. for about 45 minutes followed by cooling to about 5° C. The suspension was stirred at about 5° C. for about 45 minutes followed by centrifuging the solid and the solid was washed with 34 liters of methanol. The wet solid obtained was subjected to spin drying for about 30 minutes followed by aerial drying for about 30 minutes. The semidried solid was further dried at about 70° C. over about 11 hours to afford 81 kg of the title compound.

Purity: 99.54% by chiral HPLC.

Specific optical rotation (SOR) [α]_{D}²⁰=+90.62° (C=1% methanol).

### EXAMPLE 3

### Preparation of 2-Thienylethyl Para-Toluenesulphonate (Formula VII) Using Toluene

400 liters of toluene and 163.2 kg of para-toluene sulfonyl chloride were charged into a clean and dry reactor followed by cooling to about 5° C. 100 kg of thiophene-2-ethanol was added at about 5° C. over about 20 minutes followed by addition of 130 kg of triethylamine over about 8 hours, 50 minutes. The reaction mixture temperature was raised to about 30° C. followed by stirring for about 12 hours. The reaction mass was filtered through a Nutsche filter and washed with 2×100 liters of toluene. The reaction filtrate was transferred into another reactor followed by washing with 5×200 liters of water. Organic and aqueous layers were separated and the organic layer was distilled completely at about below 70° C. under vacuum to afford 212 kg (yield: 96.37%) of title compound.

Purity by GC: 95.59%.

### EXAMPLE 4

### Preparation of (+)-Methyl-α-(2-Thienyl Ethyl Amino)(2-Chloro Phenyl)Acetate Hydrochloride (Formula VII) Using Toluene

240 liters of demineralised water, 240 liters of dichloromethane and 120 kg of the L-(+)-tartaric acid salt of α-amino-(2-chlorophenyl)-acetic acid methyl ester of Formula III were charged into a clean and dry reactor followed by stirring at about 30° C. for about 10 minutes. The resultant reaction solution was cooled to about 5° C. followed by adjusting the pH to about 7.46 by the addition of 900 liters of 10% sodium bicarbonate solution at about 5° C. over about 1 hour, 40 minutes. Organic and aqueous layers were separated and the aqueous layer was extracted with 2×60 liters of dichloromethane. Both the organic layers were combined and the organic layer was washed with 3×120 liters of demineralised water followed by separation of organic and aqueous layers. Organic layer was distilled completely at about 30° C. over about 3.5 hours to afford a residue of the free base of Formula II. To the residue, 138.4 liters of toluene, 179.7 kg of dipotassium hydrogen phosphate and 118.2 kg of 2-(2-thiophene)ethanol tosylate of Formula VII were charged into a clean and dry reactor followed by heating to about 92.5° C. and maintained for about 30 hours. The reaction mass was cooled to 30° C. followed by charging 203 liters of toluene and 600 liters of water. The resultant reaction suspension was stirred for about 30 minutes followed by separation of organic and aqueous layers. The aqueous layer was extracted with 72 liters of toluene followed by separation of organic and aqueous layers. Both the organic layers were combined and the total organic layer was washed with 143 liters of water. Organic and aqueous layers were separated and to the organic layer 34.2 liters of 12 N hydrochloric was charged at about 12.5° C. followed by stirring for about 30 minutes. The resultant reaction mixture was heated to about 55° C. followed by stirring for about 15 minutes. Separated solid was filtered and the solid was washed with 69 liters of toluene. The wet reaction material was transferred into a reactor followed by the charging of 420 liters of acetone and 30.4 liters of 12N hydrochloric acid. The resultant reaction suspension was heated to about 58° C. followed by stirring for about 20 minutes. The above reaction solution was cooled to about 11° C. for about 45 minutes. The solid that formed was separated in a centrifuge and the solid was washed with 60 liters of acetone. Finally the solid was subjected to spin drying for about 1.5 hours followed by drying at about 62.5° C. under vacuum for about 7 hours to afford 75 kg (yield: 63.25%) of the title compound.

Purity by HPLC: 99.53%

Specific optical rotation (SOR) [α]_{D}²⁰=+110° (C=1% methanol).

### EXAMPLE 5

### Preparation of Clopidogrel Bisulfate (Formula I)

935.2 kg of formalin (40% aqueous formaldehyde) and 167 kg of (+)-methyl-α-(2-thienyl ethyl amino)-(2-chloro phenyl)acetate hydrochloride (Formula VIII) were charged into a clean and dry reactor at about 30° C. The resultant reaction mixture was cooled to about 25° C. followed by stirring for about 24 hours. After the completion of the reaction, the reaction mass was filtered into another reactor through a leaf filter. 835 liters of dichloromethane was charged followed by cooling to about 15° C. pH of the reaction suspension was adjusted to about 7.01 by adding aqueous sodium carbonate solution (33.4 kg of sodium carbonate dissolved in 835 liters of water) over about 3 hours, 15 minutes. The organic layer was separated and the aqueous layer was extracted with 501 liters of dichloromethane. Organic and aqueous layers were separated and the aqueous layer was extracted with 334 liters of dichloromethane. The organic layers were combined and the total organic layer was washed with 3×501 liters of water. Organic and aqueous layers were separated and the organic layer was distilled completely at about 45° C. under vacuum over about 5.5 hours to afford clopidogrel free base as a residue. To the residue, 1469 liters of acetone was charged at about 30° C. followed by stirring for about 15 minutes. 8.8 kg of activated carbon and 8.8 kg of Hyflo Supercel diatomaceous earth were charged followed by stirring for about 30 minutes. The resultant reaction suspension was filtered into another reactor through leaf and cartridge filters followed by washing the filters with 167 liters of acetone. 19.87 liters of water was charged to the reaction filtrate followed by cooling to about 0° C. 23.6 liters of sulphuric acid was slowly added at about 0° C. over about 3 hours, 45 minutes followed by stirring for about 10 minutes. The resultant reaction suspension was seeded by charging of 1.67 kg of the compound of Formula I followed by stirring at about 2° C. for about 5 hours.

Separated solid was centrifuged followed by washing 167 liters of acetone. The solid obtained was subjected to spin drying for about 1 hour to afford 156 kg (wet) of the title compound. The solid obtained was subjected to air drying over about 30 minutes followed by drying at about 45° C. for about 12 hours to afford 143.16 kg of the title compound.

Purity by HPLC: 99.77% w/w.

Assay by HPLC (on dry basis): 99.1% w/w.

### EXAMPLE 6

### Racemization of R-(−)-Isomer of α-Amino-(2-Chlorophenyl)-Acetic Acid Methyl Ester from Mother Liquor into Compound of Formula V

2000 liters of mother liquors that were obtained from Example-2 containing the R-(−)-isomer of the compound of Formula IV in large amounts and traces of the S-(+)-isomer of the compound of Formula III was taken into a reactor followed by heating to a temperature of about 68° C. over a period of about 13 hours followed by distilling the solvent completely at a temperature below 75° C. The obtained residue was cooled to a temperature below 50° C. 300 liters of water was added followed by stirring for about 25 minutes and the resultant mass was cooled to about 25° C. 300 liters of dichloromethane was added to the mass followed by cooling to a temperature below 25° C. The pH of the mass was adjusted to about 7 followed by separation of organic and aqueous layers. The above process was repeated three times using dichloromethane and then all the organic layers were combined followed by the addition of 100 liters of water with simultaneous stirring for about 10 minutes. The organic layer was separated and the solvent from the organic layer was distilled completely at about 70° C. under vacuum followed by cooling to below 50° C. Finally residual solvent was removed by applying vacuum of about 600 mm Hg followed by cooling the obtained residue to a temperature of about 25° C. to afford (±)-methyl-α-amino(2-chlorophenyl)acetate of Formula V.

### EXAMPLE 7

### Determination of Impurities in Clopidogrel or its Salt

TABLE 1
HPLC conditions for determining impurities in clopidogrel or it salts.

| | |
|---|---|
| Column: | ULTRON ES-OVM 150 × 4.6 mm, 5 μm |
| | (or L57 column) |
| Flow rate: | 1.0 ml/minute |
| Column oven | 27° C. |
| temperature: | |
| Detector | 220 nm |
| wavelength: | |
| Injection volume: | 10 μl. |
| Run time: | 20 minutes |
| Buffer preparation | Dissolve 1.36 g of mono basic potassium phosphate |
| | in about 500 ml of water and diluted with water |
| | to 1000 ml |
| Mobile Phase | Degassed mixture of phosphate buffer and |
| | acetonitrile (75:25) |
| Sample preparation | Dissolve 100 mg of sample in 200 ml of methanol. |

| COMPOUND | RRT |
|---|---|
| Clopidogrel impurity C | 2.1 |
| Clopidogrel impurity B | 0.8 and 1.2 |
| Clopidogrel impurity A | 0.5 |
| Clopidogrel | 1 |

RRT is relative retention time, obtained by dividing a column retention time for a compound by the retention time for clopidogrel.

### EXAMPLE 8

### Preparation of Clopidogrel Bisulfate Crystalline Form I

35 g of (+)-methyl-α-(2-thienylethylamino)-(2-chloro phenyl)acetate hydrochloride (Formula VIII) and 175 ml of formalin (37.4% aqueous formaldehyde) were charged into a clean and dry 4 neck round bottom flask followed by stirring for about 20 hours. After the completion of the reaction, the reaction mass was filtered and the filtrate was taken into a clean and dry round bottom flask. 175 ml of dichloromethane was charged followed by cooling to about 12° C. pH of the reaction suspension was adjusted to about 7.32 by adding aqueous sodium carbonate solution (8 g of sodium carbonate dissolved in 175 ml of water) over about 15 minutes. The resultant reaction suspension was stirred at about 12° C. for about 30 minutes. Organic layer was separated and the aqueous layer was extracted with 105 ml of dichloromethane followed by 70 ml of dichloromethane. Both the organic layers were combined and the total organic layer was washed with 105 ml of water. Organic and aqueous layers were separated and the organic layer was distilled completely at about 40° C. under vacuum to afford clopidogrel free base as a residue. To the residue 30 ml of 2-butanol was charged followed by cooling to about 26° C. 375 ml of 2-butanol was charged followed by stirring for about 10 minutes. 1.5 g of activated carbon was charged followed by stirring for about 10 minutes. The resultant reaction suspension was filtered through a celite bed and the celite was washed with 39 ml of 2-butanol. The filtrate was charged into a clean and dry 4 neck round followed by addition of 2.9 ml of concentrated sulphuric acid over about 10 minutes. The resultant reaction solution was seeded by charging of 0.15 g of the compound of Formula I followed by stirring at about 26° C. for about 5 hours.

Separated solid was filtered and the solid was washed with 30 ml of 2-butanol followed by washing with 15 ml of cyclohexane. The solid obtained was dried at about 95° C. for about 24 hours to afford 16.1 g of the title compound.

### EXAMPLE 9

### Preparation of Clopidogrel Bisulfate Crystalline Form I from Form II

30 g of clopidogrel bisulfate of Formula I, having crystalline Form II, and 135 ml of dichloromethane were charged into a clean and dry 4 neck round bottom flask followed by cooling to about 3° C. pH of the suspension was adjusted to about 7.0 by the addition of 10% aqueous sodium carbonate solution (8 g of sodium carbonate dissolved in 80 ml of water) over about 15 minutes. The resultant suspension was stirred at about 2° C. for about 30 minutes. The organic layer was separated and the aqueous layer was extracted with 2×70 ml of dichloromethane. Both the organic layers were combined and the total organic layer was washed with 2×45 ml of water. Organic and aqueous layers were separated and the organic layer was distilled completely at about 40° C. under vacuum to afford clopidogrel freebase as a residue. To the residue 30 ml of 2-butanol was charged followed by cooling to about 26° C. 375 ml of 2-butanol was charged followed by stirring for about 10 minutes. 1.5 g of activated carbon was charged followed by stirring for about 10 minutes. The resultant reaction suspension was filtered through celite and the celite was washed with 39 ml of 2-butanol. The reaction filtrate was charged into a clean and dry 4 neck round bottom flask followed by addition of 2.9 ml of concentrated sulphuric acid over about 10 minutes. The resultant reaction solution was seeded by charging of 0.15 g of the compound of Formula I followed by stirring at about 26° C. for about 5 hours. Separated solid was filtered and the solid was washed with 30 ml of 2-butanol followed by washing with 15 ml of cyclohexane. The solid obtained was dried at about 95° C. for about 24 hours to afford 15.9 g of clopidogrel bisulfate having crystalline Form I.

### EXAMPLE 10

### Preparation of 2-(2-Thiophene)Ethanol Tosylate (Formula VII) Using Dichloromethane

4 liters of dichloromethane was added into a reactor at a temperature of about 30° C., cooled to a temperature of about 7.5° C. to which was then added 1.784 kg of p-toluene sulphonyl chloride followed by 1 kg of thiophene-2-ethanol. 1.302 kg of triethylamine was added to the above reaction mass at a temperature of about 7.5° C. followed by slowly raising the temperature of the reaction mass to 22.5° C. for about 5 hours. The obtained reaction mass was filtered through a pressure Nutsche filter, washed with methylene chloride (2×1 liter) and the mother liquor was collected and transferred into another reactor. The organic layer was washed with water (5×2 liters). The organic layer thus obtained was subjected to distillation at a temperature below 70° C. using hot water circulation. The obtained residue was then cooled to about 30° C. to afford 2.1 kg (yield: 95.5%) of title compound.

### EXAMPLE 11

### Preparation of (+)-Methyl α-(2-Thienyl Ethyl Amino)(2-Chloro Phenyl)Acetate Hydrochloride (Formula VIII) Using Tertiary-Butyl Acetate

1.80 liters of tertiary-butyl acetate and 134.10 kg of dipotassium hydrogen phosphate were to S-(+)-isomer of methyl α-amino-(2-chlorophenyl)acetate of Formula II obtained from Example 4 at a temperature of about 30° C. 0.892 kg of 2-(2-thiophene)ethanol tosylate of Formula VII obtained from Example 5 were then added and the reaction mass was subjected to heating to a temperature of about 92.5° C. and maintained for about 24 hours followed by distilling of the solvent at a temperature below 60° C. under a vacuum of about 650 mm Hg. The obtained reaction mass was cooled to a temperature of about 12.5° C. and 2.52 liters of ethyl acetate and 2.52 liters of water were added. The mass was subjected to stirring for about 10 minutes and the solution was allowed to settle for about 15 minutes, followed by separating the aqueous layer from organic layer. The aqueous layer was extracted with ethyl acetate (2×1.8 liters). The organic layer was washed with demineralised water (3×1.12 liters). Organic layer was cooled to a temperature of about 12.5° C., and 280 ml of 36% aqueous hydrochloric acid was slowly added. The reaction mixture was stirred for about 25 minutes. The obtained reaction mass was filtered, and the solid was washed with 560 ml of ethyl acetate and subjected to spin-drying. The wet solid was transferred into a reactor followed by the addition of 2.24 liters of ethyl acetate and the mass was subjected to heating to a temperature of about 77° C. for about 75 minutes. The mass was cooled to 27.5° C. over a period of about 75 minutes, filtered and the solid washed with 560 ml of ethyl acetate. Finally the solid was subjected to spin drying followed by drying at a temperature of about 62.5° C. under vacuum of about 600 mm Hg over a period of 4 hours and cooled to a temperature of about 30° C. to afford 65 kg (yield: 66.3%) of title compound.

We claim:

1. A process for preparing clopidogrel or a salt thereof, comprising reacting racemic methyl alpha-amino-(2-chlorophenyl)acetate with L-(+)-tartaric acid, separating a formed tartaric acid salt of S-(+)-methyl alpha-amino-(2-chlorophenyl)acetate from a reaction mixture, and heating a reaction mixture to form racemic methyl alpha-amino-(2-chlorophenyl)acetate.

2. The process of claim 1, wherein a starting material methyl α-amino-(2-chlorophenyl)acetic acid is prepared by reacting DL-2-chlorophenylglycine with methanol in the presence of sulfuric acid.

3. The process of claim 1, wherein reacting with L-(+)-tartaric acid occurs in the presence of a solvent consisting essentially of methanol.

4. The process of claim 1, wherein heating a reaction mixture is carried out at temperatures about 30° C. to about 100° C.

5. The process of claim 1, wherein formed racemic methyl alpha-amino-(2-chlorophenyl)acetate is reacted with additional L-(+)-tartaric acid to form a tartaric acid salt of S-(+)-methyl alpha-amino-(2-chlorophenyl)acetate.

6. The process of claim 1, further comprising reacting a tartaric acid salt of S-(+)-methyl alpha-amino-(2-chlorophenyl)acetate with a base to form S-(+)-methyl alpha-amino-(2-chlorophenyl)acetate, which is reacted with 2-thienyl-p-toluenesulfonate to form a reaction product, and further reacting a reaction product with formaldehyde to form clopidogrel.

7. The process of claim 6, wherein 2-thienyl-p-toluenesulfonate is prepared by reacting thiophene-2-ethanol with a p-toluene sulfonyl halide, in a solvent comprising toluene.

8. The process of claim 6, wherein a base comprises sodium hydroxide, potassium hydroxide, sodium carbonate, potassium carbonate, sodium bicarbonate, or potassium bicarbonate.

9. The process of claim 6, wherein formaldehyde is used in the form of an aqueous solution having a concentration about 40 percent by weight, in a volume about 3 L to about 15 L per kg of S-(+)-methyl α-(2-thienylethylamino)-(2-chlorophenyl)acetate.

10. The process of claim 6, further comprising reacting clopidogrel with sulfuric acid to form clopidogrel hydrogen sulfate.

11. The process of claim 10, wherein clopidogrel hydrogen sulfate has crystalline Form I.

12. The process of claim 10, wherein clopidogrel hydrogen sulfate has crystalline Form II.

13. A continuous process for preparing clopidogrel or a salt thereof, comprising reacting racemic methyl alpha-amino-(2-chlorophenyl)acetate with L-(+)-tartaric acid, separating a formed tartaric acid salt of S-(+)-methyl alpha-amino-(2-chlorophenyl)acetate from a reaction mixture, heating a reaction mixture to form racemic methyl alpha-amino-(2-chlorophenyl)acetate, and reacting formed racemic methyl alpha-amino-(2-chlorophenyl)acetate with additional L-(+)-tartaric acid to form a tartaric acid salt of S-(+)-methyl alpha-amino-(2-chlorophenyl)acetate.

14. The process of claim 13, wherein reacting with L-(+)-tartaric acid occurs in the presence of a solvent consisting essentially of methanol.

15. The process of claim 13, wherein heating a reaction mixture is carried out at temperatures about 30° C. to about 100° C.

16. The process of claim 13, further comprising reacting a tartaric acid salt of S-(+)-methyl alpha-amino-(2-chlorophenyl)acetate with a base to form S-(+)-methyl alpha-amino-(2-chlorophenyl)acetate, which is reacted with 2-thienyl-p-toluenesulfonate to form a reaction product, and further reacting a reaction product with formaldehyde to form clopidogrel.

17. The process of claim 16, wherein 2-thienyl-p-toluenesulfonate is prepared by reacting thiophene-2-ethanol with a p-toluene sulfonyl halide, in a solvent comprising toluene or dichloromethane.

18. A process for preparing 2-thienyl-p-toluenesulfonate, comprising reacting thiophene-2-ethanol with a p-toluene sulfonyl halide, in a solvent comprising toluene or dichloromethane.

19. The process of claim 18, wherein reacting is conducted in the presence of a base.

20. The process of claim 18, wherein reacting is conducted in the presence of triethylamine.
